(19) United States
(12) Patent Application Publication
(10) Pub. No.: US 2024/0130731 A1
(43) Pub. Date: Apr. 25, 2024

(54) TOROID BALLOON FOR HEMORRHAGE CONTROL

(71) Applicant: CCJ MEDICAL DEVICES LLC, Raleigh, NC (US)

(72) Inventors: Colin T. BRAHMSTEDT, Colorado Springs, CO (US); John B. HOLCOMB, Houston, TX (US); Christopher J. MURPHY, Asheville, NC (US)

(73) Assignee: CCJ MEDICAL DEVICES LLC, Raleigh, NC (US)

(21) Appl. No.: 18/277,235

(22) PCT Filed: Feb. 14, 2022
(86) PCT No.: PCT/US2022/016298

§ 371 Date: Aug. 14, 2023

Related U.S. Application Data
(60) Provisional application No. 63/149,522, filed on Feb. 15, 2021.
(60) Provisional application No. 63/149,523, filed on Feb. 15, 2021.

(51) Int. Cl. A61B 17/12; A61B 5/0215

(52) U.S. Cl. CPC A61B 17/12036; A61B 5/0215; A61B 17/12109; A61B 17/12136; A61B 2017/12004; A61M 25/1002

(57) ABSTRACT

An occlusion device includes a balloon and a catheter. The occlusion device is configured to allow blood to pass through the balloon, instead of around the balloon. By allowing the blood to pass through the balloon, constant traction against the vessel wall is achieved, regardless of flow rate, thereby mitigating distal migration. The balloon includes an orifice to allow blood flow through the balloon. The orifice is independent of the diameter of the vessel, therefore the effect of the diameter of the vessel changing during the procedure will not affect the flow rate of blood. The occlusion device enables finer titration of blood flow by varying the size of the orifice when compared to conventional solutions.

### SUMMARY

Disclosed herein are implementations of an occlusion device and systems for measuring blood pressure using the occlusion device. In an aspect, an occlusion device includes a catheter and a toroidal balloon. The catheter may include a lumen. The toroidal balloon may be coupled to the catheter. The toroidal balloon may include an outer surface that has a first diameter. The outer surface may be configured to seal against an inner portion of a vessel wall. The toroidal balloon may include an inner lumen that has a second diameter. The inner lumen may be configured to allow blood to flow through the toroidal balloon.

In one or more aspects, the toroidal balloon may be inflatable via the lumen to dynamically decrease the second diameter to decrease the blood flow through the toroidal balloon. In one or more aspects, the toroidal balloon may be deflatable via the lumen to dynamically increase the second diameter to increase the blood flow through the toroidal balloon. In one or more aspects, the outer surface may include one or more traction elements to reduce migration of the occlusion device. In one or more aspects, the catheter may be coupled to the toroidal balloon such that the catheter passes through a body of the toroidal balloon. In one or more aspects, the catheter may be coupled to the toroidal balloon such that the catheter runs tangent to the inner lumen. In one or more aspects, the toroidal balloon may be comprised of at least one of a silicone material, a polyurethane material, a latex material, a polyether block amide (PEBA) material, a nylon material, a polyethylene terephthalate (PET) material, or any suitable material.

In one or more aspects, the occlusion device may include a second toroidal balloon coupled to the catheter. In one or more aspects, the catheter may comprise a second lumen that is configured to inflate and deflate the second toroidal balloon. In one or more aspects, the second toroidal balloon may be comprised of at least a silicone material, a polyurethane material, a latex material, a PEBA material, a nylon material, or a PET material.

In one or more aspects, the occlusion device may include a third toroidal balloon coupled to the catheter. In one or more aspects, the catheter may comprise a third lumen that is configured to inflate and deflate the third toroidal balloon. In one or more aspects, the third toroidal balloon may be comprised of at least a silicone material, a polyurethane material, a latex material, a PEBA material, a nylon material, or a PET material. In one or more aspects, the third toroidal balloon may be configured to provide rigidity to the occlusion device.

In one or more aspects, the catheter may include a port and a flexible membrane. The flexible membrane may be coupled to the port. The flexible membrane may be configured to act as a barrier to translate the pressure from the vessel into the fluid filled lumen, which may be measured by a pressure sensor to determine the blood pressure. In one or more aspects, the port may be disposed on a proximal side of the toroidal balloon and the flexible membrane may be configured to measure blood pressure on the proximal side of the toroidal balloon. In one or more aspects, the catheter may include a second port and a second flexible membrane. The second flexible membrane may be coupled to the second port. The second flexible membrane may be configured to measure blood pressure. In one or more aspects, the second port may be disposed on a distal side of the toroidal balloon and the second flexible membrane may be configured to measure blood pressure on the distal side of the toroidal balloon.

In one or more aspects, the catheter may include a micro electro-mechanical system (MEMS) blood pressure sensor configured to measure blood pressure. In one or more aspects, the MEMS blood pressure sensor may be disposed on a proximal side of the toroidal balloon and configured to measure blood pressure on the proximal side of the toroidal balloon. In one or more aspects, the catheter may include a second MEMS blood pressure sensor disposed on a distal side of the toroidal balloon and configured to measure blood pressure on the distal side of the toroidal balloon.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIGS. 1A and 1B show a conventional balloon catheter for resuscitative endovascular balloon occlusion of the aorta (REBOA) and partial REBOA (p-REBOA), respectively.

FIGS. 2A and 2B show an example of a balloon in accordance with the embodiments of this disclosure.

FIGS. 3A and 3B are diagrams of examples of a single toroidal balloon mounted to a catheter in accordance with embodiments of this disclosure.

FIGS. 4A and 4B are diagrams of examples of a single toroidal balloon mounted to a catheter in accordance with embodiments of this disclosure.

FIG. 5 is a diagram of a perspective view of an example of a two tandem balloon catheter in accordance with embodiments of this disclosure.

FIG. 6 is a diagram of a side view of an example of a toroidal balloon that is configured to prevent migration of the balloon.

FIG. 7 is a diagram of a perspective view of an example of a hybrid toroidal balloon.

FIG. 8 is a diagram of a perspective view of an example of a two concentric toroidal balloon catheter in accordance with embodiments of this disclosure,

FIGS. 9A and 9B are diagrams of examples of a multi-staged toroidal balloon mounted to a catheter.

FIG. 10 is a diagram of an example of a system for blood pressure sensing.

FIG. 11 is a diagram of an example of a system for sampling differential pressures across an occlusion in a vessel.

### DETAILED DESCRIPTION

Non-compressible torso hemorrhage (NCTH) has been identified as a leading cause of potentially survivable death from trauma in both battlefield and civilian settings. A standard therapeutic option to increase systolic blood pressure and maintain cardiac and cerebral perfusion in these patients is aortic occlusion. Traditionally performed through open surgical aortic cross-clamping, the less invasive method of REBOA has been developed, to minimize subsequent surgical trauma and related complications. However, an undesired side effect of full aortic occlusion is rapid and permanent ischemic damage to downstream organs and tissue. Recent studies have shown that by performing a partial occlusion, proximal systemic blood pressure can be maintained, while providing partial blood flow to the organs and tissue below the balloon, reducing risk for temporary or permanent ischemic damage. Current REBOA solutions with spherical balloons to titrate blood flow around the partial occlusion have proven challenging to maintain, requiring constant monitoring and careful manipulation.

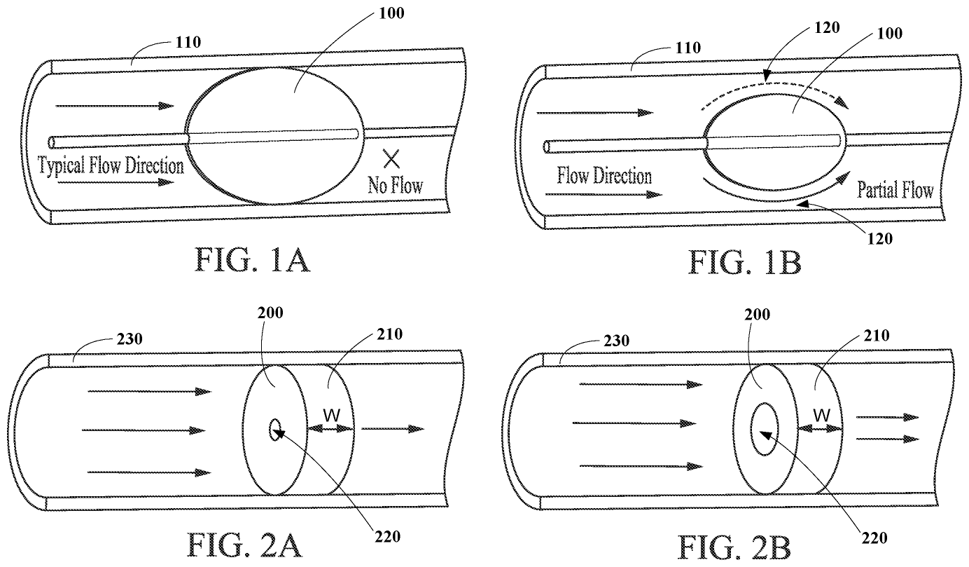

FIGS. 1A and 1B show a conventional balloon catheter 100 for REBOA and p-REBOA, respectively. The example shown in FIG. 1A is a 20 mm balloon catheter in a 20 mm vessel 110. It is understood that vessel diameter and balloon catheter diameter can vary, and 20 mm is shown merely as an example. Conventional REBOA solutions perform an aortic occlusion by inserting a balloon catheter into the aorta through the femoral artery and positioning the balloon onto either Zone 1 or Zone 3 of the aorta. The balloon catheter 100 is inflated until full occlusion is reached, as shown in FIG. 1A. Attempts to create p-REBOA are made by partially deflating the balloon catheter 100, as shown in FIG. 1B. In the example shown in FIG. 1B, the balloon catheter 100 is deflated, for example, to 18 mm in the 20 mm vessel to attempt to achieve partial occlusion by allowing blood to bypass around the exterior of the balloon catheter 100.

It is critical that flow rates be consistent and smooth, as abrupt changes to flow or pressure increase the chance of rupturing any therapeutic clots that may have formed below the balloon. In conventional methods such as the example shown in FIG. 1B, a gap 120 between the balloon catheter 100 and the wall of the vessel 110 must be carefully controlled to maintain the desired flow rate. This is extremely difficult to achieve due to the vascular elasticity, changing hemodynamics, and a compliant balloon. For example, to maintain a therapeutic target flow rate of 0.5 L/min, the gap required should be less than 1 mm. Additionally, deflating the balloon causes the balloon to lose contact with the vessel wall, and thus lose traction that secures the balloon in place and results in distal migration in the vessel.

By way of contrast to conventional REBOA and p-REBOA solutions, the embodiments disclosed herein allow blood to pass through the balloon, instead of around the balloon. By allowing the blood to pass through the balloon, constant traction against the vessel wall may be achieved, regardless of flow rate, thereby mitigating distal migration. The embodiments disclosed herein include an orifice to allow blood flow through the balloon. The orifice is independent of the diameter of the vessel, therefore the effect of the diameter of the vessel changing during the procedure will not affect the flow rate of blood, in contrast with the conventional REBOA and p-REBOA solutions. The embodiments disclosed herein enable easier titration of blood flow by varying the size of the orifice when compared to conventional REBOA and p-REBOA solutions. The embodiments disclosed herein enable tight control of optimal distal flow and pressure, which minimizes rebleeding and distal ischemia while mitigating distal migration.

In the embodiments disclosed herein, a toroidal balloon, when inflated with either a gas or a sterile fluid, is configured to inflate and stretch its outer diameter to seal against a vessel wall, leaving an inner lumen (e.g., an orifice) for blood to pass through. The toroidal balloon may be constructed of a compliant material, a non-compliant material, a semi-compliant material, or a hybrid material that includes at least one compliant portion and at least one non-compliant portion. These materials may include, but are not limited to silicone, polyurethane, latex, polyether block amide (PEBA), nylon, and polyethylene terephthalate (PET).

FIGS. 2A and 2B show an example of a toroidal partial occlusion balloon 200 in accordance with the embodiments of this disclosure. The toroidal partial occlusion balloon 200 includes an outer surface 210 and an inner lumen 220. The outer surface 210 is configured to seal against a vessel wall 230 when the toroidal partial occlusion balloon 200 is inflated. The toroidal partial occlusion balloon may have a width W that ranges from approximately 10 mm to 30 mm. FIG. 2A shows the toroidal partial occlusion balloon 200 configured for a low flow state. The arrows indicate the direction and effect on the flow. FIG. 2B shows a toroidal partial occlusion balloon 200 configured for a medium flow. As shown in FIGS. 2A and 2B, the flow is greater in the balloon shown in FIG. 2B when compared to the flow of the balloon shown in FIG. 2A. As shown in FIGS. 2A and 2B, the flow is based on the area of the inner lumen 220 such that a larger area provides a greater flow. The example balloons shown in FIGS. 2A and 2B may be mounted on a catheter to create a fixed or adjustable fluid restriction size. The catheter may have at least one lumen used to inflate the attached balloon. The catheter may have additional lumens for other balloon configurations, blood pressure measurements, blood sampling and interventional use cases.

Since flow at a static pressure differential is linearly proportional to a cross-sectional area, relocating the locus of the flow path to the center of the balloon provides finer and more predictable control of blood flow, as area changes less for a given increment at smaller radii. The following example contrasts the radius changes required to establish a flow rate of 0.48 L/min with a pressure of 90 mmHg proximal to the balloon and a pressure of 40 mmHg distal to the balloon.

The mass flow rate qₘ across the inner lumen 220 may be calculated as follows:

$${q}_{m} = \frac{{C}_{d}}{\sqrt{1 - {β}^{4}}} ∈ \frac{π}{4} {d}^{2} \sqrt{2 Δ p * {ρ}_{1}}$$

where C_{d} is the coefficient of discharge, β is a diameter ratio of inner lumen diameter d to vessel diameter D, ϵ is an expansibility factor (1 for incompressible gases and most liquids, and decreasing with pressure ratio across the inner lumen), d is the inner lumen diameter under operating conditions, ρ₁ is a fluid density in plane of upstream tapping, and Δp is a differential pressure measured across the inner lumen 220.

Referring to FIG. 1B, the balloon catheter 100 may be deflated such that the outer diameter of the balloon catheter is 19.9 mm and the gap 120 between the outer surface of the balloon and the inner surface of the vessel wall may be 0.05 mm. In this example, since the vessel diameter is 20 mm, the cross-sectional area of the gap 120 is 3.14 mm². In order to achieve the same cross-sectional area (i.e., 3.14 mm²) for the toroidal partial occlusion balloon 200 shown in FIG. 2B, the diameter of the inner lumen 220 is adjusted to 2 mm by inflating or deflating the toroidal partial occlusion balloon 200.

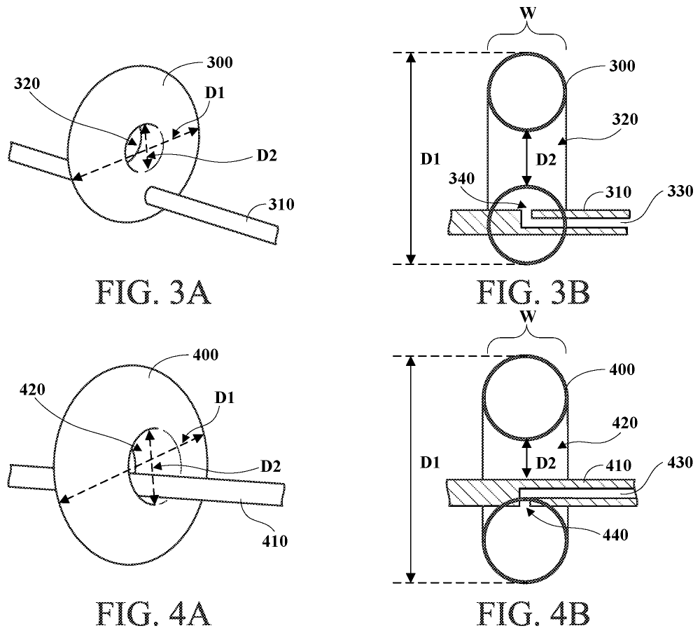

FIGS. 3A and 3B are diagrams of examples of a single toroidal balloon 300 mounted to a catheter 310 in accordance with embodiments of this disclosure. FIG. 3A shows a perspective view of the single toroidal balloon 300 mounted to the catheter 310, and FIG. 3B shows a cross-sectional view of the single toroidal balloon 300 mounted to the catheter 310. As shown in FIG. 3A, the single toroidal balloon 300 has an outer diameter D1 and an inner lumen 320. The single toroidal balloon 300 is configured to inflate to increase the outer diameter D1 to form a seal between the outer surface of the single toroidal balloon 300 and an inner surface of a vessel such that the single toroidal balloon 300 is securely held in place within the vessel. Once the seal is formed, further inflation can be used to adjust the flow of blood. The inner lumen 320 has a diameter D2 that can be varied to adjust the flow of blood, for example, by inflating the single toroidal balloon 300 (e.g., after the seal is formed) to reduce the flow of blood (e.g., by reducing the diameter D2 of the inner lumen 320) and deflating the single toroidal balloon 300 to increase the flow of blood (e.g., by increasing the diameter D2 of the inner lumen 320). In some examples, the single toroidal balloon 300 may be inflated such that the diameter D2 is reduced to zero to provide a total occlusion of the vessel. As shown in FIG. 3B, the single toroidal balloon 300 has a width W. In some embodiments, the width W may be increased such that the surface area of the single toroidal balloon 300 is increased, which may result in a reduction of localized pressure on a vessel wall. In this example, the single toroidal balloon 300 may be mounted to the catheter 310 such that the body of the catheter 310 passes through the body of the single toroidal balloon 300 as shown in FIGS. 3A and 3B. The single toroidal balloon 300 may be secured to the catheter by an ultraviolet cure adhesive, a room temperature vulcanization, a cyanoacrylate, a medical grade adhesive, mechanical coupling, ultrasonic welding, laser welding, or any suitable securing method.

The catheter 310 may be a single or multi-lumen catheter. In an example, the catheter size may be 3Fr-12Fr (1 mm-4 mm outer diameter). The catheter 310 may have a single lumen 330 to support inflation of the single toroidal balloon 300 as shown in FIG. 3B, multiple lumens to support inflation of multiple toroidal balloons, or it may have multiple lumens for additional accessory use cases. Example additional accessory use cases include, and are not limited to, blood pressure measurements, blood sampling, biomarker measurements such as lactate, blood pH, and the like, and interventional use cases such as delivering medication, fluids, or blood products. The single lumen 330 includes an opening 340 to the interior of the body of the single toroidal balloon 300. The opening 340 allows for passage of fluid (e.g., liquid or gas) to inflate and deflate the single toroidal balloon 300. In addition to REBOA and p-REBOA applications, the example single toroidal balloon 300 shown in FIGS. 3A and 3B may be used to treat truncal hemorrhage and for treatment of cardiac arrest.

FIGS. 4A and 4B are diagrams of examples of a single toroidal balloon 400 mounted to a catheter 410 in accordance with embodiments of this disclosure. FIG. 4A shows a perspective view of the single toroidal balloon 400 mounted to the catheter 410, and FIG. 4B shows a cross-sectional view of the single toroidal balloon 400 mounted to the catheter 410. As shown in FIG. 4A, the single toroidal balloon 400 has an outer diameter D1 and an inner lumen 420. The inner lumen 420 has a diameter D2 that can be varied to adjust the flow of blood, for example, by inflating the single toroidal balloon 400 to reduce the flow of blood. (e.g., by reducing the diameter D2 of the inner lumen 420) and deflating the single toroidal balloon 400 to increase the flow of blood (e.g., by increasing the diameter D2 of the inner lumen 420). As shown in FIG. 4B, the balloon has a width W. In some embodiments, the width W may be increased such that the surface area of the single toroidal balloon 400 is increased, which may result in a reduction of localized pressure on a vessel wall. In this example, the single toroidal balloon 400 may be mounted such that the catheter 410 runs tangent to an inner surface of the inner lumen 420 as shown in FIGS. 4A and 4B. The single toroidal balloon 400 may be secured to the catheter 410 by an ultraviolet cure adhesive, a room temperature vulcanization, a cyanoacrylate, a medical grade adhesive, mechanical coupling, ultrasonic welding, laser welding, or any suitable securing method.

The catheter 410 may be a single or multi-lumen catheter. In an example, the catheter size may be 3Fr-12Fr (1 mm-4 mm outer diameter). The catheter 410 may have a single lumen 430 to support inflation of the single toroidal balloon 400 as shown in FIG. 4B, multiple lumens to support inflation of multiple toroidal balloons, or it may have multiple lumens for additional accessory use cases. The single lumen 430 includes an opening 440 to the interior of the body of the single toroidal balloon 400. The opening 440 allows for passage of fluid to inflate and deflate the single toroidal balloon 400. In addition to REBOA and p-REBOA applications, the example single toroidal balloon 400 shown in FIGS. 4A and 4B may be used to treat truncal hemorrhage and for treatment of cardiac arrest.

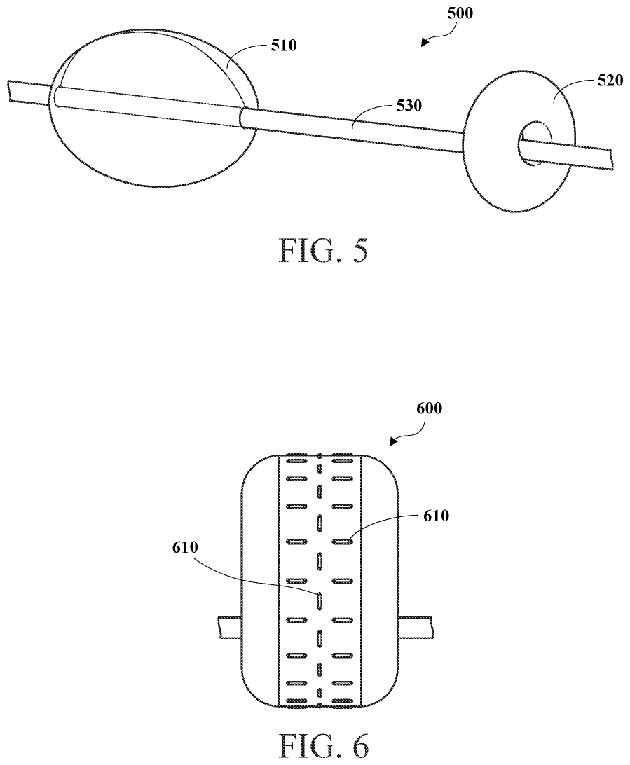

FIG. 5 is a diagram of an example of perspective view of a two tandem balloon catheter 500 in accordance with embodiments of this disclosure. The two tandem balloon catheter 500 includes a standard balloon 510, such as the balloon 100 shown in FIGS. 1A and 1B, and a toroidal balloon 520, such as any one of the balloons 200-400 shown in FIGS. 2A-4B. In this example, the standard balloon 510 may be used to provide total occlusion and the toroidal balloon 520 may be used to provide partial occlusion. In this example, the catheter 530 may be a multi-lumen catheter that includes a first lumen to inflate/deflate the standard balloon 510 and a second lumen to inflate/deflate the toroidal balloon 520.

FIG. 6 is a diagram of an example of a side view of a toroidal balloon 600 that is configured to prevent migration of the toroidal balloon 600 in a vessel. The toroidal balloon 600, may be any one of the balloons 200-400 shown in FIGS. 2A-4B and toroidal balloon 520 shown in FIG. 5. As shown in this example, the toroidal balloon 600 includes one or more traction elements 610. The traction elements 610 are configured to provide additional grip to the vessel wall to prevent migration of the toroidal balloon 600. The material of the traction elements 610 may include, but are not limited to silicone, polyurethane, latex, PEBA, nylon, and PET. In some examples, different traction elements may be of different materials. In some examples, the material of the toroidal balloon 600 and the traction elements 610 may be the same or different.

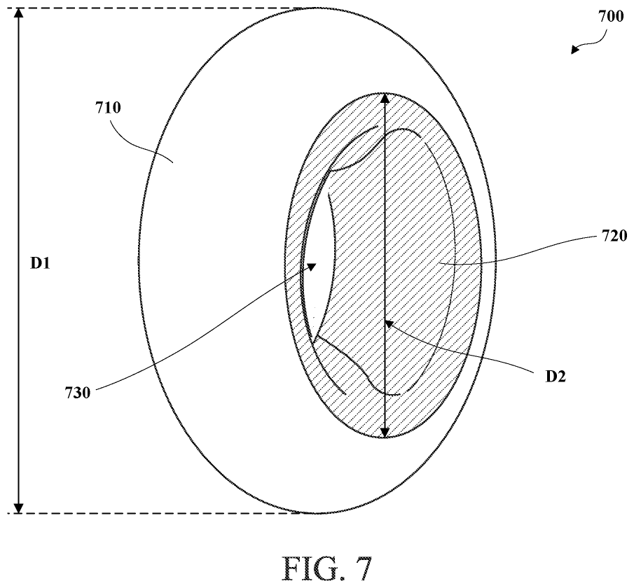

FIG. 7 is a diagram of a perspective view of an example of a hybrid toroidal balloon 700. The hybrid toroidal balloon 700 may be any one of the balloons 200-400 shown in FIGS. 2A-4B, toroidal balloon 520 shown in FIG. 5, and toroidal balloon 600 shown in FIG. 6. As shown in FIG. 7, the hybrid toroidal balloon 700 includes a first portion 710 (unshaded) and a second portion 720 (shaded). The first portion 710 is associated with an outer diameter D1 of the hybrid toroidal balloon 700 that is configured to contact the vessel wall when inflated. The second portion 720 is associated with an inner diameter D2 of the hybrid toroidal balloon 700 that is configured to allow blood to flow through an inner lumen 730. In order to promote the ability to maintain a fixed inner lumen while being able to expand and fill a vessel wall, the hybrid toroidal balloon 700 may be made of a combination of materials with compliant and non-compliant properties. In this example, the first portion 710 may be made of a compliant material, and the second portion 720 may be made of a non-compliant material. In another example, the first portion 710 may be made of a non-compliant material, and the second portion 720 may be made of a compliant material.

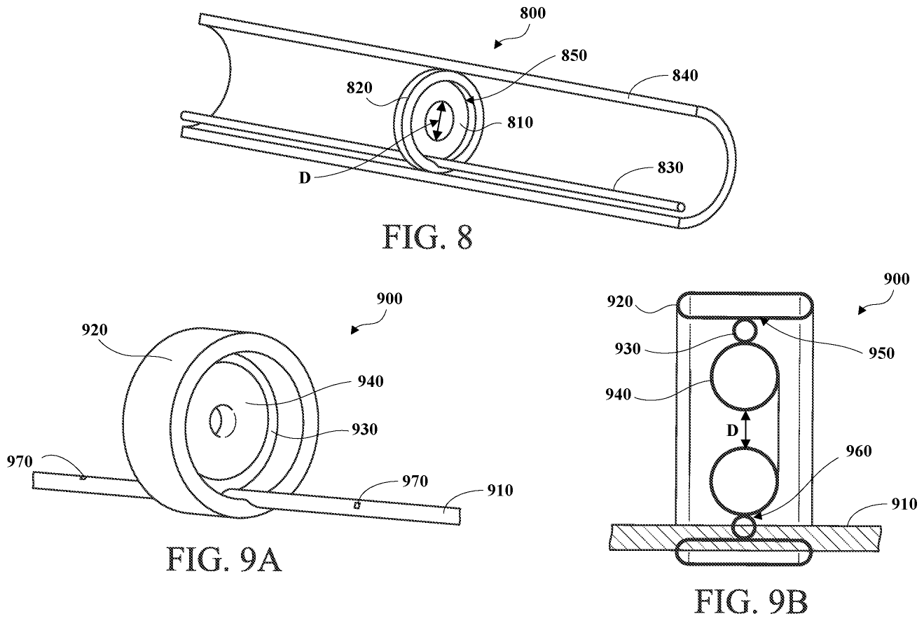

FIG. 8 is a diagram of a perspective view of an example of a two concentric toroidal balloon catheter 800 in accordance with embodiments of this disclosure. The two concentric toroidal balloon catheter 800 includes a first toroidal balloon 810, a second toroidal balloon 820, and a catheter 830. In this example, the two concentric toroidal balloon catheter 800 is shown in a vessel 840. The first toroidal balloon 810 is configured to provide a reliable and predictable shape with a known inner lumen diameter D. The first toroidal balloon 810 includes an outer perimeter 850 that is attached to a surface of an inner lumen of the second toroidal balloon 820. The second toroidal balloon 820, when inflated, is configured to provide a seal between the vessel 840 and the first toroidal balloon 810. The second toroidal balloon 820 may be configured to stretch and accommodate a broad range of vessel diameters by maintaining positive pressure against the vessel wall while the first toroidal balloon 810 is configured to inflate to adjust the area of the inner lumen of the first toroidal balloon 810 to provide the desired partial flow. In this example, the catheter 830 is a multi-lumen catheter that includes a first lumen that is configured to inflate/deflate the first toroidal balloon 810 and a second lumen that is configured to inflate/deflate the second toroidal balloon 820.

In some embodiments, the two concentric toroidal balloon catheter 800 may be configured as a fixed flow device. In one example, the first toroidal balloon 810 may be replaced with a balloon that when inflated, will provide a reliable and predictable shape with a known lumen diameter. The balloon may be configured for a variety of fixed inner lumen sizes. The fixed inner lumen sizes may range from 1 mm to 4 mm. In particular, some example fixed inner lumen sizes may be 2 mm, 2.5 mm, or 3 mm.

FIGS. 9A and 9B are diagrams of examples of a multi-staged toroidal balloon 900 mounted to a catheter 910. FIG. 9A shows a perspective view of the multi-staged toroidal balloon 900 mounted to the catheter 910, and FIG. 9B shows a cross-sectional view of the multi-staged toroidal balloon 900 mounted to the catheter 910. As shown in FIGS. 9A and 9B, the multi-staged toroidal balloon 900 includes three nested toroidal balloons. In these examples, the multi-staged toroidal balloon 900 includes a first balloon 920, a second balloon 930, and a third balloon 940. Three nested toroidal balloons are shown as an example, and some embodiments may include more than three nested toroidal balloons.

Each of the first balloon 920, the second balloon 930, and the third balloon 940 may be toroidal balloons. The first balloon 920 may be a compliant balloon such that it is in contact with the vessel wall when inflated to provide a seal against the vessel wall. Due to varying human anatomy, the first balloon 920 is configured to stretch to seal against a wide variety of vessel sizes. The second balloon 930 may be a non-compliant balloon that has an outer perimeter that is attached to a surface of an inner lumen 950 of the first balloon 920. The second balloon 930 is configured to act as a spine to provide some rigidity to the multi-staged toroidal balloon 900. The third balloon 940 may be a compliant balloon that has an outer perimeter that is attached to a surface of an inner lumen 960 of the second balloon 930. The third balloon 940, when inflated, is configured to press against the surface of the inner lumen 960 of the second balloon 930 to inflate and decrease the diameter D of the inner lumen of the third balloon 940, thereby allowing for variable orifice sizes. The variable orifice sizes enable variable and controllable restrictions to blood flow in a blood vessel. Such flow rates may vary from 0 L/min to 4 L/min. The first balloon 920, the second balloon 930, and the third balloon 940 may be connected to a multi-lumen catheter, such as the catheter 910, that provides independent control of each balloon volume. In this example, the catheter 910 includes blood pressure sensors 970 on either sides of the multi-staged toroidal balloon to monitor pressures and transmit alerts when a flow control balloon (e.g., the third balloon) requires an adjustment.

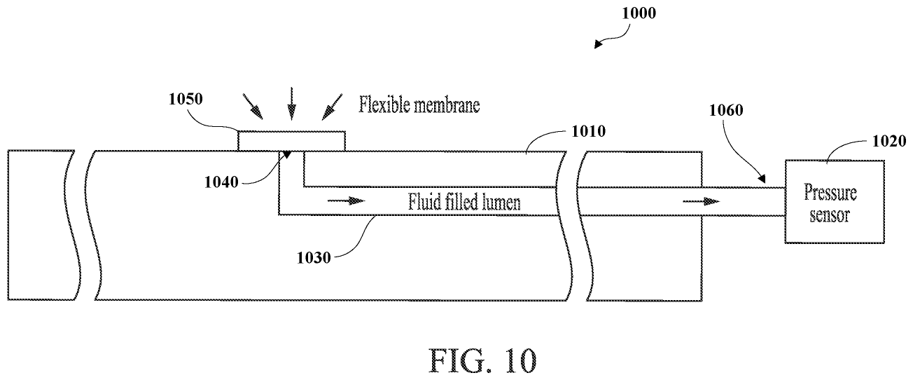

FIG. 10 is a diagram of an example of a system 1000 for blood pressure sensing. As shown in FIG. 10, the system 1000 includes a multi-lumen catheter 1010 and an external pressure sensor 1020. For simplicity and clarity, the multi-lumen catheter 1010 is shown with on lumen 1030, The multi-lumen catheter includes at least one port 1040 covered with a flexible membrane 1050. The multi-lumen catheter may include multiple ports that are each covered with a flexible membrane, and one port is shown in FIG. 10 for simplicity and clarity. Each port of the multi-lumen catheter is associated with its own lumen. Each lumen may be filled with a fluid. Each lumen may have a distal end 1060 that is connected to the external pressure sensor 1020. Blood pressure causes the flexible membranes to flex, thereby pushing the fluid within the lumen 1030 towards the external pressure sensor 1020. The external pressure sensor 1020 detects the pressure caused by the movement of the fluid and translates the detected pressure into a pressure reading. The pressure reading may be displayed on a display of the pressure sensor or an external device, such as a mobile phone or some other connected device.

The system 1000 allows the pressure sensing mechanism to be fully sealed and avoids the need to prime the lumens before use, thereby enabling easier deployment in the field, as well as reducing the risk of clot formation in the catheter 1010, These lumens may be primed during manufacture, and the sensors may be built directly into the disposable catheter. In some embodiments, the membrane separation layer may be excluded to enable a user to manually prime the lumens. In some embodiments, the fluid filled lumens may be replaced with micro electro-mechanical system (MEMS) blood pressure sensors embedded in the catheter 1010 to measure the blood pressure directly.

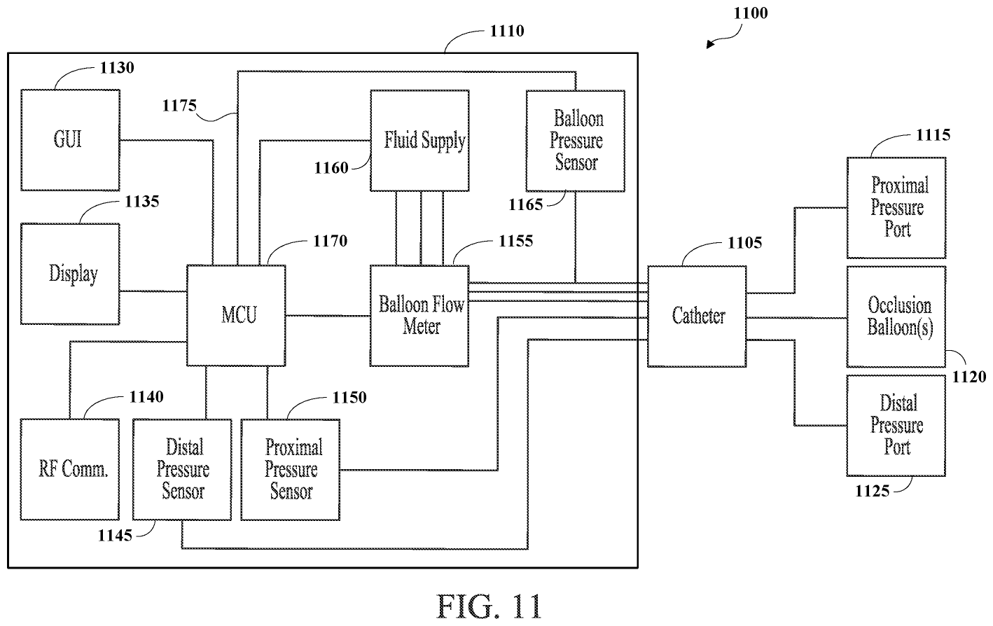

FIG. 11 is a diagram of an example of a system 1100 for sampling differential pressures across an occlusion in a vessel. In addition, the system 1100 may be configured to create a blood flow restriction with a toroidal balloon, such as any of the toroidal balloons described above, dynamically adjust the diameter of the inner lumen of the toroidal balloon in response to readings from one or more sensors, or both. The system 1100 includes a catheter 1105 and a controller 1110. The catheter 1105 may be a disposable catheter and the controller 1110 may be a reusable control unit. In some examples, the system 1100 may be an integrated system such that the catheter 1105 and controller 1110 are both disposable.

The catheter 1105 includes a proximal pressure port 1115, one or more occlusion balloons 1120, and a distal pressure port 1125. The one or more occlusion balloons 1120 may include any of the balloons described above. The proximal pressure port 1115, the distal pressure port 1125, or both, may be configured with a flexible membrane, such as the flexible membrane 1050 shown in FIG. 10. Alternatively, the proximal pressure port 1115, the distal pressure port 1125, or both, may be replaced with a MEMS blood pressure sensor embedded in the catheter 1105 to measure the blood pressure directly.

The controller 1110 includes a graphical user interface (GUI) 1130, a display 1135, a radio frequency (RF) communications unit 1140, a distal pressure sensor 1145, a proximal pressure sensor 1150, a balloon flow meter 1155, a fluid supply 1160, a balloon pressure sensor 1165, and a microcontroller unit (MCU) 1170. The GUI 1130, the display 1135, the RF communications unit 1140, the distal pressure sensor 1145, the proximal pressure sensor 1150, the balloon flow meter 1155, the fluid supply 1160, and the balloon pressure sensor 1165 may be in communication with the MCU 1170 via a bus 1175. The controller 1110 may be designed for fixed or portable use.

The GUI 1130 may be configured to obtain user inputs and may be controlled by software performing real-time analysis of sensor inputs and system response. The GUI 1130 may display, on the display 1135, real-time blood pressure readings above and below the occlusion, as well as maintain a desired flow rate.

The RF communications unit 1140 may be configured to securely communicate with an external device for remote patient monitoring. The RF communications unit 1140 may be configured to communicate using any suitable wireless technology, such as, for example, Bluetooth, near-field communications (NFC), ultra-wideband communications (UWC), WiFi, or any cellular communications such as Long Term Evolution (LTE) or 5G.

The distal pressure sensor 1145 is configured to measure blood pressure above the occlusion (e.g., distal to the balloon where blood is flowing away from the balloon) and the proximal pressure sensor 1150 is configured to measure blood pressure below the occlusion (e.g., proximal to the balloon where blood is flowing towards the balloon).

The balloon flow meter 1155 is configured to measure the flow of fluid to and from the catheter 1105. The fluid supply 1160 may include a tank that contains fluid that is used to supply the catheter 1105 during inflation of the one or more occlusion balloons 1120. In some examples, the flow meter 1155 or the fluid supply 1160 may include a pump. During deflation of the one or more occlusion balloons 1120, the fluid supply is configured to store fluid from the one or more occlusion balloons 1120.

The balloon pressure sensor 1165 is configured to detect a pressure of an occlusion balloon against a vessel wall. The system 1100 may determine that the one or more occlusion balloons 1120 are properly secured within the vessel based on the data obtained from the balloon pressure sensor 1165.

The MCU 1170 is configured to dynamically adjust the one or more occlusion balloons 1120 to maintain the target blood flow in response to varying proximal and distal hemodynamics. For example, when the proximal pressure sensor 1150 detects a blood pressure above a threshold, the MCU 1170 may determine that one or more of the occlusion balloons 1120 should be deflated to increase the size of the respective inner lumen to increase blood flow and thereby reduce the blood pressure. In this example, the MCU 1170 may deflate the one or more occlusion balloons 1120 using the balloon flow meter 1155 to control the flow of fluid from the one or more occlusion balloons 1120 to the fluid supply 1160. When the MCU 1170 determines that the desired blood pressure is achieved, for example, based on the detected blood pressure from the distal pressure sensor 1145, the proximal pressure sensor 1150, or both, the MCU 1170 will stop deflation of the one or more occlusion balloons 1120 by stopping the flow of the fluid to the fluid supply 1160. The system 1100 may include a manual operation mode that allows a user to bypass automated controls and set the one or more occlusion balloons 1120 to a fixed size.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

1. An occlusion device, comprising:
a catheter comprising a catheter lumen; and
a toroidal balloon coupled to the catheter, wherein the toroidal balloon comprises:
an outer surface having a first diameter and configured to seal against an inner portion of a vessel wall; and
an inner lumen configured to allow blood to flow therethrough and comprising:
an adjustable second diameter adapted to be varied by a user from at least a first inner lumen diameter enabling a first blood flow rate to a second inner lumen diameter enabling a second blood flow rate.

2. The occlusion device of claim 1, wherein the inner lumen of the toroidal balloon is inflatable via the catheter lumen to dynamically decrease the second diameter from the first inner lumen diameter to the second inner lumen diameter to decrease the blood flow rate through the inner lumen from a first blood flow rate to a second blood flow rate less than the first blood flow rate.

3. The occlusion device of claim 1, wherein the toroidal balloon is deflatable via the catheter lumen to dynamically increase the second diameter from the first inner lumen diameter to the second inner lumen diameter to increase the blood flow through the inner lumen.

4. The occlusion device of claim 1, wherein the outer surface includes one or more traction elements to reduce migration of the occlusion device.

5. The occlusion device of claim 1, wherein the catheter is coupled to the toroidal balloon such that the catheter passes through a body of the toroidal balloon.

6. The occlusion device of claim 1, wherein the catheter is coupled to the toroidal balloon such that the catheter runs tangent to the inner lumen.

7. The occlusion device of claim 1, wherein at least a portion of the toroidal balloon is comprised of at least one of a silicone material, a polyurethane material, a latex material, a polyether block amide (PEBA) material, a nylon material, or a polyethylene terephthalate (PET) material.

8. The occlusion device of claim 1, wherein the toroidal balloon comprises:
a first toroidal balloon comprising:
the outer surface, wherein the outer surface is adapted to seal against the inner portion of a vessel wall; and
an inner surface; and
a second toroidal balloon concentrically coupled to the inner surface of the first toroidal balloon, the second toroidal balloon comprising the inner lumen and the adjustable second diameter.

9. The occlusion device of claim 8, wherein the catheter comprises a second catheter lumen, wherein the second catheter lumen is configured to inflate and deflate the second toroidal balloon.

10. (canceled)

11. The occlusion device of claim 1, wherein the toroidal balloon comprises:
a first toroidal balloon comprising:
the outer surface, wherein the outer surface is adapted to seal against the inner portion of a vessel wall; and
an inner diameter;
a second toroidal balloon concentrically coupled to the inner diameter of the first toroidal balloon and comprising a second inner diameter; and
a third toroidal balloon concentrically coupled to the second inner diameter, the third toroidal balloon comprising the inner lumen and the adjustable second diameter.

12. The occlusion device of claim 11, wherein the catheter further comprises:
a second catheter lumen configured to inflate and deflate the second toroidal balloon; and
a third catheter lumen configured to inflate and deflate the third toroidal balloon.

13. The occlusion device of claim 11, wherein at least one of the first and second toroidal balloon is configured to provide rigidity to the occlusion device.

14. (canceled)

15. The occlusion device of claim 1, further comprising:
at least a first blood pressure measurement element selected from:
a micro-electro mechanical system (MEMS) blood pressure sensor; and
a first port having a first flexible membrane coupled thereto and configured to measure blood pressure.

16. The occlusion device of claim 15, wherein the catheter further comprises:
at least a second blood pressure measurement element selected from:
a micro-electro mechanical system (MEMS) blood pressure sensor; and
a second port having a second flexible membrane coupled thereto and configured to measure blood pressure;
wherein one of the first blood pressure measurement element and the second blood pressure measurement element measures blood pressure proximal to the toroidal balloon and the other of the first blood pressure measurement element and the second blood pressure measurement element measures blood pressure distal to the toroidal balloon.

17-20. (canceled)

21. The occlusion device of claim 1, further comprising:
at least a first blood pressure measurement element adapted to measure at least one of a blood pressure proximal to toroidal the balloon and a blood pressure distal to the toroidal balloon,
wherein the adjustable second diameter is adapted to be varied by a user to establish one of a desired blood pressure proximal to the toroidal balloon and a desired blood pressure distal to the toroidal balloon.

22. The occlusion device of claim 21, wherein the adjustable second diameter is adapted to be varied by a user to establish one of a blood pressure of about 90 mm Hg proximal to the toroidal balloon and a blood pressure of about 40 mm Hg distal to the toroidal balloon.

23. An occlusion device, comprising:
a catheter comprising a catheter lumen;
a toroidal balloon coupled to the catheter, wherein the toroidal balloon comprises:
an outer surface having a first diameter and configured to seal against an inner portion of a vessel wall; and
an inner lumen configured to allow blood to flow therethrough and comprising an adjustable second diameter adapted to be varied by a user; and
at least a first blood pressure measurement element adapted to measure at least one of a blood pressure proximal to the balloon and a blood pressure distal to the balloon, wherein the adjustable second diameter is adapted to be varied by a user to establish one of a desired blood pressure proximal to the balloon and a desired blood pressure distal to the balloon.

24. An occlusion system, comprising:
a catheter comprising a catheter lumen;
a toroidal balloon coupled to the catheter, wherein the toroidal balloon comprises:
an outer surface having a first diameter and configured to seal against an inner portion of a vessel wall; and
an inner lumen configured to allow blood to flow therethrough and comprising an adjustable second diameter adapted to be varied by one of a user and a controller to achieve
at least one of a desired blood flow and a desired blood pressure;
at least a first blood pressure measurement element adapted to measure at least one of a blood pressure proximal to the toroidal balloon and a blood pressure distal to the toroidal balloon; and
a controller for varying the adjustable second diameter to achieve at least one of a desired blood flow within the range of 0-4 L/min, a desired blood pressure proximal to the toroidal balloon, and a desired blood pressure distal to the toroidal balloon.

25. The occlusion system of claim 24, wherein the controller is adapted to vary the adjustable second diameter to achieve one of a blood pressure proximal to the toroidal balloon of about 90 mm Hg and a blood pressure distal to the toroidal balloon of about 40 mm Hg.
